(19) United States
(12) Patent Application Publication
(10) Pub. No.: US 2026/0337301 A1
(43) Pub. Date: Oct. 29, 2026

(54) METHODS AND COMPOSITIONS USEFUL FOR FRACTIONATING BIOMASS USING A SCHIFF-BASE IONIC LIQUID

(71) Applicants: The Regents of the University of California, Oakland, CA (US); National Technology & Engineering Solutions of Sandia, LLC, Albuquerque, NM (US)

(72) Inventors: Mood Mohan, Oak Ridge, TN (US); Hemant Choudhary, Emeryville, CA (US); Venkataramana R. Pidatala, Emeryville, CA (US); Blake A. Simmons, San Francisco, CA (US); Seema Singh, Clarksburg, MD (US); John M. Gladden, Alameda, CA (US)

(21) Appl. No.: 19/114,410

(22) PCT Filed: Sep. 19, 2023
(86) PCT No.: PCT/US2023/033180

§ 371 Date: Mar. 21, 2025

Related U.S. Application Data
(60) Provisional application No. 63/376,575, filed on Sep. 21, 2022.

(51) Int. Cl. C08B 37/00; C08H 7/00

(52) U.S. Cl. CPC C08B 37/0003; C08B 37/0009; C08B 37/0057; C08H 6/00

(57) ABSTRACT

The present invention provides for a method to deconstruct a biomass: the method comprising: introducing a solvent comprising a Schiff-base ionic liquid (SBIL) to a biomass, such that the solvent solubilizes at least a part of the biomass to form a solubilized biomass mixture.

### CROSS-REFERENCE TO RELATED APPLICATIONS

This application claims priority to U.S. Provisional Patent Application Ser. No. 63/376,575, filed on Sep. 21, 2022, which is hereby incorporated by reference.

### STATEMENT OF GOVERNMENTAL SUPPORT

The invention was made with government support under Contract Nos. DE-AC02-05CH11231 awarded by the U.S. Department of Energy. The government has certain rights in the invention.

### FIELD OF THE INVENTION

The present invention is in the field of biomass pretreatment.

### BACKGROUND OF THE INVENTION

Lignocellulosic biomass is a renewable feedstock that can be used to produce fuels and value-added chemicals. The conversion of lignocellulosic biomass is a demanding task since water and many traditional organic solvents are not effectively deconstructing the biomass due to its structural arrangement of its constituent biopolymers (cellulose, hemicellulose, and lignin). Hence, it requires a suitable pretreatment solvent for delignification and thereby improves the enzymatic accessibility to convert biomass into fuel and platform chemicals. The discovery of alternative solvents such as ionic liquids (ILs) has mitigated the issues involved in the processing of biomass to some extent. To date, many of the ILs studied for biomass pretreatment suffer from high viscosity, high cost, low thermal stability, and high melting point. Due to these factors, new ILs that can overcome these issues need to be discovered.

Depleting fossil-derived organic solvents are still widely used in daily and industrial activities, especially synthetic chemistry, despite the extensive literature describing related environmental, health, and safety issues.1 In this regard, ionic liquids (ILs; organic cation(s) containing salts with a melting point below 100 °C) has been identified as a promising alternative to organic solvents.2 Given the ease of tunability, ILs can be designed to accommodate several advantages including chemical and thermal stability, dissolution ability, negligible vapor pressure, among others.3,4,5 Due to this, ILs have found applications in several areas, for example, catalysis,6,7 biologically actives,8,9 process development,10 energy storage,5,11 energy dense materials,12 biomedical,13 lubricants,14 and others.15 Although not all reported ILs are renewable, cost-effective, biodegradable and non-toxic, the potential of limitless combinations of cation(s) and anion(s) facilitates design and production of distinct ILs with unique physicochemical and desired properties to meet the specific application.

Among several examples mentioned above, ILs have been found to be exceptional in biopolymer dissolution that facilitates their bioconversion into biofuels.16,17,18,19,20,21 Application of IL technologies for sustainable processing of biomass to meet the large quantities of biofuels, demands a reliable and renewable source for IL productions. Unfortunately, majority of the ILs produced today relies on fast depleting fossil resources restricting the exploration of huge potential offered by billions of tons of unused available lignocellulosic biomass feedstock that includes agricultural-, forest-, and herbaceous residues. The design of new renewable solvents, particularly ILs, from renewable sources thereby remain an open quest for researchers in the field.

Lignin is an underutilized source of aromatics and is largely utilized for generating heat and power in a (bio)refinery and pulp & paper industries though combustion. Lignin, primarily consisting of phenylpropanoids, can be effectively oxidized to produce aldehydes such as vanillin and syringaldehyde.22,23,24,25 These aldehydes can undergo condensation reactions with amines to afford imines (also known as Schiff base) that could be further protonated to afford range of suitable ILs for various applications. Previous efforts from our lab have focused on the reductive amination of biomass-derived aldehydes to synthesize renewable ILs that were shown to be very effective for lignocellulosic pretreatment and demonstrated a concept of close-loop biorefinery.26

### SUMMARY OF THE INVENTION

The present invention provides for a method to deconstruct a biomass: the method comprising: introducing a solvent comprising a Schiff-base ionic liquid (SBIL) to a biomass, such that the solvent solubilizes at least a part of the biomass to form a solubilized biomass mixture.

In some embodiments, the method further comprises separating a solubilized biomass from the SBIL; optionally introducing an enzyme and/or a microbe to the solubilized biomass such that the enzyme and/or microbe produces a sugar from the solubilized biomass; and, (c) optionally separating the sugar from the solubilized biomass.

In some embodiments, the method further comprises forming the SBIL prior to the introducing step, and optionally forming the solvent by providing the SBIL.

The present invention provides for a composition comprising: (a) a solvent comprising a Schiff-base ionic liquid (SBIL), and (b) a biomass.

In some embodiments, the SBIL comprises a C═N moiety formed by condensation of an aldehyde or ketone and an amine. In some embodiments, the SBIL is an azomethine.

In some embodiments, the solvent comprises an ionic liquid (IL) that is not a Schiff-base, and/or a deep eutectic solvent (DES).

The present invention provides for a method to improve the biomass deconstruction efficacy utilizing lignin-derived Schiff-base ionic liquids (SBILs). In some embodiments, the SBIL is an organic salt with an iminium ion (formed by the protonation of the imine obtained after the reaction of an aldehyde or ketone with an amine). Depending on the specific acid or base being utilized for making SBILs, the physio-chemical properties such as low melting point, low viscosity, and high thermal stability can be achieved, which are desired features in an IL for efficient biomass pretreatment. The advantage of the SBILs is the enabling of the cost-effective production of fermentable sugars and lignin removal which is a major hurdle for producing commercially viable bioenergy from waste biomass.

The present invention provides for a solvent for deconstructing biomass involving the use of Schiff-base ionic liquids. Schiff-bases have been widely investigated in biological and pharmaceutical research fields and the molecules having azomethine Schiff-base skeleton are clinically approved drugs. Interestingly, SBILs have not been employed for biomass pretreatment and processing. The present invention provides for using SBILs as a solvent to improve the biomass deconstruction efficacy given the unique charge delocalization on C═N moiety. Depending on the type of anion or cation supported to Schiff-base, several key properties can be leveraged related to their performance as effective pretreatment solvents.

Preliminarily, two different Schiff-bases containing lignin-derived vanillin and ethylenediamine at different molar ratios (1:1 and 2:1) are synthesized. After the synthesis of Schiff-bases, we prepared three SBILs by mixing acetic acid to these two Schiff-bases at 1:1 and 2:1 molar ratio. Biomass (sorghum) pretreatment experiments were conducted to study the efficacy of prepared SBILs as a function of sugar yields. The saccharification of SBIL-pretreated biomass (20 wt % biomass and 80 wt % IL loadings) released up to 88% glucose and 76% xylose. Additional work will be carried out to optimize the pretreatment process in terms of screening of SBILs, reaction conditions, lignin removal, sugar yields, and possible recycling, etc. In some embodiments, the SBIL comprise a vanillin and/or an ethylenediamine. In some embodiments, the vanillin and the ethylenediamine have a molar ratio of about 1:1 to about 2:1. In some embodiments, the percent biomass and the percent IL (including the SBIL) by weight are about X wt % and Y wt %, respectfully, wherein X plus Y equals 100. In some embodiments, X is 1, 5, 10, 15, 20, 25, 30, 40, or 50, or any value between any two preceding values; and Y is 99, 95, 90, 85, 80, 75, 70, 60, or 50, or any value between any two preceding values.

In some embodiments, the present invention is used to convert waste biomass (from agricultural residues, wood/paper/pulping, grasses) into biofuels and/or bioproducts. In some embodiments, the process helps achieve a high concentration of fermentable sugars while leaving the residual lignin for valuable chemicals.

Advantages of the present invention may include one or more of the following: (1) SBILs can be developed from renewable and inexpensive resources such as lignin. (2) Highly compatible with downstream processes owing to biocompatibility and biodegradability properties. (3) Versatile with various biomass types. (4) Ability to tune the SBILs properties (low melting point, viscosity, better interactions, etc.) (5) Easy synthesis of lignin-derived ILs; no tedious derivatization. (6) Unsaturated bonds (double bond) could be effective in the removal of lignin due to the π-staking and reduce the viscosity. (7) Properties of SBILs could be different and unique compared to other common ionic liquids (1-ethyl-3-methylimidazolium acetate) and protic ionic liquids (ethanolamine acetate).

In some embodiments, the method further comprises ensiling a biomass, prior to the introducing step, to produce an ensiled biomass comprising one or more organic acids, wherein the ensile biomass is the biomass of the introducing step. In some embodiments, the ensiled biomass comprises equal to or more than about 10%, 20%, 30%, or 40% by weight of the one or more organic acids. In some embodiments, the one or more organic acids comprises an alkanoic acid. In some embodiments, the alkanoic acid is lactic acid, acetic acid, butyric acid, or propionic cid, or a mixture thereof. In some embodiments, the ensiling step produces one or more toxic compounds in the ensiled biomass, and the microbe is resistant to the one or more toxic compounds. In some embodiments, the one or more toxic compound is an organic acid, such as a straight chained or branched alkanoic acid (such as acetic acid, lactic acid, or formic acid), or an aromatic organic acid (such as benzoic acid, vanillic acid, or the like). In some embodiments, the organic acid has between about 2 to 10 carbon atoms.

In some embodiments, the method further comprises one or more steps taught in U.S. Provisional Patent Application Ser. No. 63/016,877, filed Apr. 28, 2020, and U.S. patent application Ser. No. 17/242,256, filed Apr. 27, 2021 (both are hereby incorporated by reference in their entireties.

In some embodiments, the method further comprises (b) introducing an enzyme and/or a microbe to the solubilized biomass mixture such that the enzyme and/or microbe produces a sugar from the solubilized biomass mixture. In some embodiments, the method further comprises (c) separating the sugar from the solubilized biomass mixture.

The present invention provides for compositions and methods described herein. In some embodiments, the compositions and methods further comprise steps, features, and/or elements described in U.S. patent application Ser. No. 16/737,724, hereby incorporated by reference in its entirety.

In some embodiments, SBIL, IL and/or DES are bio-compatible.

The present invention provides for compositions and methods described herein.

In some embodiments, the compositions and methods further comprise steps, features, and/or elements described in U.S. patent application Ser. No. 16/737,724, hereby incorporated by reference in its entirety.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. FT-IR profiles of vanillin (black), ethylene diamine (gray), and as-synthesized Schiff base 1 (red) and 2 (blue).

FIG. 2. FT-IR profiles of acetic acid (dark gray), as-synthesized Schiff base 1 (red) or 2 (blue), 1:1 acetate IL 1A or 2A (olive), and 1:2 acetate IL 1B or 2B (cyan).

FIG. 3. Differential scanning calorimetry and thermal gravimetric analysis plots of Schiff bases 1 (red) and 2 (blue) and respective ILs; 1:1 Schiff base:acetate (olive; 1A or 2A) and 1:2 Schiff base:acetate (cyan; 1B or 2B).

FIG. 4. Activity coefficients of cellulose (dark gray) and lignin (gray) in IL 1A, 1B, and 2B.

FIG. 5. (left) Powder X-ray diffraction, and (right) thermal gravimetric analysis of untreated (black) and IL 2B pretreated (gray) sorghum biomass.

FIG. 6. HSQC NMR profile of untreated (left) and pretreated (right) sorghum.

FIG. 7. Synthetic scheme of Schiff bases and related ILs.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” when applied to a value, describes a value that includes up to 10% more than the value described, and up to 10% less than the value described.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

In some embodiments, the introducing step takes place in a vessel and homogenized. In some embodiments, the loading is solid loading and controlled at about 5%, 10%, 15%, 20%, 25%, 30%, 35%, or 40%, or a range within any two preceding values. In some embodiments, the biomass and the solvent are heated, such as to 100 °C, 110 °C, 120 °C, 130 °C, 140 °C, 150 °C, 160 °C, 170 °C, 180 °C, 190 °C, 200 °C, 200 °C, 212 °C, or a range within any two preceding values, for a period of time, such as about 1 h, 2 h, 3 h, 4 h, or 5 h, or a range within any two preceding values. In some embodiments, after pretreatment, the mixture is cooled, such as for a period of about at least 30 mins, such as at room temperature, or about 25 °C, and/or then washed at least about 1×, 2×, 3×, 4×, or 5× with water, such as deionized water. In some embodiments, the resulting solid is recovered, such as separating the solid portion with the liquid portion.

In some embodiments, the biomass is a lignocellulosic biomass. In some embodiments, the vessel is made of a material that is inert, such as stainless steel or glass, that does not react or interfere with the reactions in the pretreatment mixture.

In some embodiments, the method further comprises heating the mixture, optionally also comprising the enzyme and/or microbe, to a temperature that is equal to, about, or near the optimum temperature for the enzymatic activity of the enzyme and/or growth of the microbe. In some embodiments, the enzyme is a genetically modified host cell capable of converting the cellulose in the biomass into a sugar. In some embodiments, there is a plurality of enzymes. In some embodiments, the microbe is a genetically modified host cell capable of converting a sugar produced from the biomass into a biofuel, bioproduct and/or chemical compound. In some embodiments, there is a plurality of microbes. In some embodiments, the method produces a sugar and a lignin from the biomass. The sugar is used for growth by the microbe.

In some embodiments, the solubilizing is full, near full (such as at least about 70, 80, or 90°/), or partial (such as at least about 10, 20, 30, 40, 50, or 60%). In some embodiments, the mixture is a slurry.

### Ionic Liquid

Ionic liquids (ILs) are salts that are liquids rather than crystals at room temperatures. It will be readily apparent to those of skill that numerous ILs can be used in the present invention. In some embodiments of the invention, the IL is suitable for pretreatment of the biomass and for the hydrolysis of cellulose by thermostable cellulase. Suitable ILs are taught in ChemFiles (2006) 6(9) (which are commercially available from Sigma-Aldrich, Milwaukee, Wis.). Such suitable ILs include, but are not limited to, 1-alkyl-3-alkylimidazolium alkanate, 1-alkyl-3-alkylimidazolium alkylsulfate, 1-alkyl-3-alkylimidazolium methylsulfonate, 1-alkyl-3-alkylimidazolium hydrogensulfate, 1-alkyl-3-alkylimidazolium thiocyanate, and 1-alkyl-3-alkylimidazolium halide, wherein an “alkyl” is an alkyl group comprising from 1 to 10 carbon atoms, and an “alkanate” is an alkanate comprising from 1 to 10 carbon atoms. In some embodiments, the “alkyl” is an alkyl group comprising from 1 to 4 carbon atoms. In some embodiments, the “alkyl” is a methyl group, ethyl group or butyl group. In some embodiments, the “alkanate” is an alkanate comprising from 1 to 4 carbon atoms. In some embodiments, the “alkanate” is an acetate. In some embodiments, the halide is chloride.

In some embodiments, the IL includes, but is not limited to, 1-ethyl-3-methylimidazolium acetate (EMIN Acetate), 1-ethyl-3-methylimidazolium chloride (EMIN Cl), 1-ethyl-3-methylimidazolium hydrogensulfate (EMIM HOSO₃), 1-ethyl-3-methylimidazolium methylsulfate (EMIM MeOSO₃), 1-ethyl-3-methylimidazolium ethylsulfate (EMIM EtOSO₃), 1-ethyl-3-methylimidazolium methanesulfonate (EMIM MeSO₃), 1-ethyl-3-methylimidazolium tetrachloroaluminate (EMIM AlCl₄), 1-ethyl-3-methylimidazolium thiocyanate (EMIM SCN), 1-butyl-3-methylimidazolium acetate (BMIM Acetate), 1-butyl-3-methylimidazolium chloride (BMIM Cl), 1-butyl-3-methylimidazolium hydrogensulfate (BMIM HOSO₃), 1-butyl-3-methylimidazolium methanesulfonate (BMIM MeSO₃), 1-butyl-3-methylimidazolium methylsulfate (BMIM MeOSO₃), 1-butyl-3-methylimidazolium tetrachloroaluminate (BMIM AlCl₄), 1-butyl-3-methylimidazolium thiocyanate (BMIM SCN), 1-ethyl-2,3-dimethylimidazolium ethylsulfate (EDIM EtOSO₃), Tris(2-hydroxyethyl)methylammonium methylsulfate (MTEOA MeOSO₃), 1-methylimidazolium chloride (MIM Cl), 1-methylimidazolium hydrogensulfate (MIM HOSO₃), 1,2,4-trimethylpyrazolium methylsulfate, tributylmethylammonium methylsulfate, choline acetate, choline salicylate, and the like.

In some embodiments, the ionic liquid is a chloride ionic liquid. In other embodiments, the ionic liquid is an imidazolium salt. In still other embodiments, the ionic liquid is a 1-alkyl-3-imidazolium chloride, such as 1-ethyl-3-methylimidazolium chloride or 1-butyl-3-methylimidazolium chloride.

In some embodiments, the ionic liquids used in the invention are pyridinium salts, pyridazinium salts, pyrimidium salts, pyrazinium salts, imidazolium salts, pyrazolium salts, oxazolium salts, 1,2,3-triazolium salts, 1,2,4-triazolium salts, thiazolium salts, isoquinolium salts, quinolinium salts isoquinolinium salts, piperidinium salts and pyrrolidinium salts. Exemplary anions of the ionic liquid include, but are not limited to halogens (e.g., chloride, floride, bromide and iodide), pseudohalogens (e.g., azide and isocyanate), alkyl carboxylate, sulfonate, acetate and alkyl phosphate.

Additional ILs suitable for use in the present invention are described in U.S. Pat. Nos. 6,177,575; 9,765,044; and, 10,155,735; U.S. Patent Application Publication Nos. 2004/0097755 and 2010/0196967; and, PCT International Patent Application Nos. PCT/US2015/058472, PCT/US2016/063694, PCT/US2017/067737, and PCT/US2017/036438 (all of which are incorporated in their entireties by reference). It will be appreciated by those of skill in the art that others ILs that will be useful in the process of the present invention are currently being developed or will be developed in the future, and the present invention contemplates their future use. The ionic liquid can comprise one or a mixture of the compounds.

In some embodiments, the IL is a protic ionic liquid (PIL). Suitable protic ionic liquids (PILs) include fused salts with a melting point less than 100 °C with salts that have higher melting points referred to as molten salts. Suitable PPILs are disclosed in Greaves et al. “Protic Ionic Liquids: Properties and Applications” Chem. Rev. 108(1):206-237 (2008). PILs can be prepared by the neutralization reaction of certain Brønsted acids and Brønsted bases (generally from primary, secondary or tertiary amines, which are alkaline) and the fundamental feature of these kinds of ILs is that their cations have at least one available proton to form hydrogen bond with anions. In some embodiments, the protic ionic liquids (PILs) are formed from the combination of organic ammonium-based cations and organic carboxylic acid-based anions. PILs are acid-base conjugate ILs that can be synthesized via the direct addition of their acid and base precursors. In some embodiments, the PIL is a hydroxyalkylammonium carboxylate. In some embodiments, the hydroxyalkylammonium comprises a straight or branched C1, C2, C3, C4, C5, C6, C7, C8, C9, or C10 chain. In some embodiments, the carboxylate comprises a straight or branched C1, C2, C3, C4, C5, C6, C7, C8, C9, or C10 chain. In some embodiments, the carboxylate is substituted with one or more hydroxyl groups. In some embodiments, the PIL is a hydroxyethylammonium acetate.

In some embodiments, the protic ionic liquid (PIL) is disclosed by U.S. Patent Application Publication No. 2004/0097755, hereby incorporated by reference.

Suitable salts for the method include combinations of organic ammonium-based cations (such as ammonium, hydroxyalkylammonium, or dimethylalkylammonium) with organic carboxylic acid-based anions (such as acetic acid derivatives (C1-C8), lactic acid, glycolic acid, and DESs such as ammonium acetate/lactic acid).

Suitable IL, such as distillable IL, are disclosed in Chen et al. “Distillable Ionic Liquids: reversible Amide O Alkylation”, Angewandte Comm. 52:13392-13396 (2013), King et al. “Distillable Acid-Base Conjugate Ionic Liquids for Cellulose Dissolution and Processing”, Angewandte Comm. 50:6301-6305 (2011), and Vijayaraghavan et al. “C02-based Alkyl Carbamate Ionic Liquids as Distillable Extraction Solvents”, ACS Sustainable Chem. Engin. 2:31724-1728 (2014), all of which are hereby incorporated by reference.

Suitable PIL, such as distillable PIL, are disclosed in Idris et al. “Distillable Protic Ionic Liquids for Keratin Dissolution and Recovery”, ACS Sustainable Chem. Engin. 2:1888-1894 (2014) and Sun et al. “One-pot integrated biofuel production using low-cost biocompatible protic ionic liquids”, Green Chem. 19(13):3152-3163 (2017), all of which are hereby incorporated by reference.

In some embodiments, the PILs are formed with the combination of organic ammonium-based cations and organic carboxylic acid-based anions. PILs are acid-base conjugate ILs that can be synthesized via the direct addition of their acid and base precursors. Additionally, when sufficient energy is employed, they can dissociate back into their neutral acid and base precursors, while the PILs are re-formed upon cooling. This presents a suitable way to recover and recycle the ILs after their application. In some embodiments, the PIL (such as hydroxyethylammonium acetate-[Eth][OAc]) is an effective solvent for biomass pretreatment and is also relatively cheap due to its ease of synthesis (Sun et al., Green Chem. 19(13):3152-3163 (2017)).

### Deep Eutectic Solvent (DES)

DESs are systems formed from a eutectic mixture of Lewis or Brønsted acids and bases which can contain a variety of anionic and/or cationic species. DESs can form a eutectic point in a two-component phase system. DESs are formed by complexation of quaternary ammonium salts (such as, choline chloride) with hydrogen bond donors (HBD) such as amines, amides, alcohols, or carboxylic acids. The interaction of the HBD with the quaternary salt reduces the anion-cation electrostatic force, thus decreasing the melting point of the mixture. DESs share many features of conventional ionic liquid (IL), and promising applications would be in biomass processing, electrochemistry, and the like. In some embodiments, the DES is any combination of Lewis or Brønsted acid and base. In some embodiments, the Lewis or Brønsted acid and base combination used is distillable.

In some embodiments, DES is prepared using an alcohol (such as glycerol or ethylene glycol), amines (such as urea), and an acid (such as oxalic acid or lactic acid). The present invention can use renewable DESs with lignin-derived phenols as HBDs. Both phenolic monomers and phenol mixture readily form DES upon heating at 100 °C with specific molar ratio with choline chloride. This class of DES does not require a multistep synthesis. The DES is synthesized from lignin which is a renewable source.

Both monomeric phenols and phenol mixture can be used to prepare DES. DES is capable of dissolving biomass or lignin, and can be utilized in biomass pretreatment and other applications. Using DES produced from biomass could lower the cost of biomass processing and enable greener routes for a variety of industrially relevant processes.

The DES, or mixture thereof, is bio-compatible: meaning the DES, or mixture thereof, does not reduce or does not significantly reduce the enzymatic activity of the enzyme, and/or is not toxic, and/or does not reduce or significantly reduce, the growth of the microbe. A “significant” reduction is a reduction to 70, 80, 90, or 95% or less of the enzyme's enzymatic activity and/or the microbe's growth (or doubling time), if the DES, or mixture thereof, was not present.

In some embodiments, the DES, or mixture thereof, comprises a quaternary ammonium salt and/or glycerol. In some embodiments, the DES, or mixture thereof, comprises a quaternary ammonium salt and/or glycerol. In some embodiments, the quaternary ammonium salt and/or glycerol have a molar ratio of about 1:1 to about 1:3. In some embodiments, the quaternary ammonium salt and/or glycerol have a molar ratio of about 1:1.5 to about 1:2.5. In some embodiments, the quaternary ammonium salt and/or glycerol have a molar ratio of about 1:1.8 or 1:1.9 to about 1:2.1 or 1:2.2. In some embodiments, the quaternary ammonium salt and/or glycerol have a molar ratio of about 1:2. In some embodiments, the quaternary ammonium salt is a choline halide, such choline chloride.

In some embodiments, the DES is distillable if the DES can be recovered at least equal to or more than about 50%, 55%, 60%, 65%, 70%, 75%, 80%, or 85% yield by distilling over vacuum at a temperature at about 100 °C, 110 °C, 120 °C, 130 °C, 140 °C, 150 °C, or 160 °C, or any temperature between any two of the preceding temperatures.

In some embodiments, the DES can be one taught in WO 2018/204424 (Seema Singh et al.), which is hereby incorporated in its entirety by reference.

In some embodiments, the method further comprises heating the one-pot composition, optionally also comprising the enzyme and/or microbe, to a temperature that is equal to, about, or near the optimum temperature for the enzymatic activity of the enzyme and/or growth of the microbe. In some embodiments, the enzyme is a genetically modified host cell capable of converting the cellulose in the biomass into a sugar. In some embodiments, there is a plurality of enzymes. In some embodiments, the microbe is a genetically modified host cell capable of converting a sugar produced from the biomass into a biofuel and/or chemical compound. In some embodiments, there is a plurality of microbes. In some embodiments, the introducing step(s) produce a sugar and a lignin from the biomass. The lignin can further be processed to produce a DES. The sugar is used for growth by the microbe.

In some embodiments, the solubilizing is full, near full (such as at least about 70, 80, or 90°/), or partial (such as at least about 10, 20, 30, 40, 50, or 60%). In some embodiments, the one-pot composition is a slurry. When the steps described herein are continuous, the one-pot composition is in a steady state.

In some embodiments, the introducing step comprises heating the mixture comprises increasing the temperature of the solution to a value within a range of about 75 °C to about 125 °C. In some embodiments, the heating step comprises increasing the temperature of the solution to a value within a range of about 80 °C to about 120 °C. In some embodiments, the heating step comprises increasing the temperature of the solution to a value within a range of about 90 °C to about 110 °C. In some embodiments, the heating step comprises increasing the temperature of the solution to about 100 °C.

### Enzyme

In some embodiments, the enzyme is a cellulase. In some embodiments, the enzyme is thermophilic or hyperthermophilic. In some embodiments, the enzyme is any enzyme taught in U.S. Pat. Nos. 9,322,042; 9,376,728; 9,624,482; 9,725,749; 9,803,182; and 9,862,982; and PCT International Patent Application Nos. PCT/US2015/000320, PCT/US2016/063198, PCT/US2017/036438, PCT/US2010/032320, and PCT/US2012/036007 (all of which are incorporated in their entireties by reference).

### Microbe

In some embodiments, the microbe is any prokaryotic or eukaryotic cell, with any genetic modifications, taught in U.S. Pat. Nos. 7,985,567; 8,420,833; 8,852,902; 9,109,175; 9,200,298; 9,334,514; 9,376,691; 9,382,553; 9,631,210; 9,951,345; and 10,167,488; and PCT International Patent Application Nos. PCT/US14/48293, PCT/US2018/049609, PCT/US2017/036168, PCT/US2018/029668, PCT/US2008/068833, PCT/US2008/068756, PCT/US2008/068831, PCT/US2009/042132, PCT/US2010/033299, PCT/US2011/053787, PCT/US2011/058660, PCT/US2011/059784, PCT/US2011/061900, PCT/US2012/031025, and PCT/US2013/074214 (all of which are incorporated in their entireties by reference).

Generally, although not necessarily, the microbe is a yeast or a bacterium. In some embodiments, the microbe is Rhodosporidium toruloides or Pseudomonas putida. In some embodiments, the microbe is a Gram-negative bacterium. In some embodiments, the microbe is of the phylum Proteobactera. In some embodiments, the microbe is of the class Gammaproteobacteria. In some embodiments, the microbe is of the order Enterobacteriales. In some embodiments, the microbe is of the family Enterobacteriaceae. Examples of suitable bacteria include, without limitation, those species assigned to the Escherichia, Enterobacter, Azotobacter, Erwinia, Bacillus, Pseudomonas, Klebsielia, Proteus, Salmonella, Serratia, Shigella, Rhizobia, Vitreoscilla, and Paracoccus taxonomical classes. Suitable eukaryotic microbes include, but are not limited to, fungal cells. Suitable fungal cells are yeast cells, such as yeast cells of the Saccharomyces genus.

Yeasts suitable for the invention include, but are not limited to, Yarrowia, Candida, Bebaromyces, Saccharomyces, Schizosaccharomyces and Pichia cells. In some embodiments, the yeast is Saccharomyces cerevisae. In some embodiments, the yeast is a species of Candida, including but not limited to C. tropicalis, C. maltosa, C. apicola, C. paratropicalis, C. albicans, C. cloacae, C. guillermondii, C. intermedia, C. lipolytica, C. panapsilosis and C. zeylenoides. In some embodiments, the yeast is Candida tropicalis. In some embodiments, the yeast is a non-oleaginous yeast. In some embodiments, the non-oleaginous yeast is a Saccharomyces species. In some embodiments, the Saccharomyces species is Saccharomyces cerevisiae. In some embodiments, the yeast is an oleaginous yeast. In some embodiments, the oleaginous yeast is a Rhodosporidium species. In some embodiments, the Rhodosporidium species is Rhodosporidium toruloides.

In some embodiments the microbe is a bacterium. Bacterial host cells suitable for the invention include, but are not limited to, Escherichia, Corynebacterium, Pseudomonas, Streptomyces, and Bacillus. In some embodiments, the Escherichia cell is an E coli, E. albertii, E fergusonii, E. hermanii, E. marmotae, or E. vulneris. In some embodiments, the Corynebacterium cell is Corynebacterium glutamicum, Corynebacterium kroppenstedtii, Corynebacterium alimapuense, Corynebacterium amycolatum, Corynebacterium diphtheriae, Corynebacterium efficiens, Corynebacterium jeikeium, Corynebacterium macginleyi, Corynebacterium matruchotii, Corynebacterium minutissimum, Corynebacterium renale, Corynebacterium striatum, Corynebacterium ulcerans, Corynebacterium urealyticum, or Corynebacterium uropygiale. In some embodiments, the Pseudomonas cell is a P. putida, P. aeruginosa, P. chlororaphis, P. fluorescens, P. pertucinogena, P. stutzeri, P. syringae, P. cremoricolorata, P. entomophila, P. fulva, P. monteilii, P. mosselii, P. oryzihabitans, P. parafluva, or P. plecoglossicida. In some embodiments, the Streptomyces cell is a S. coelicolor, S. lividans, S. venezuelae, S. ambofaciens, S. avermitilis, S. albus, or S. scabies. In some embodiments, the Bacillus cell is a B. subtilis, B. megaterium, B. lichenmformis, B. anthracis, B. amyloliquefaciens, or B. pumilus.

### Biofuel

In some embodiments, the biofuel produced is ethanol, or any other organic molecule, described produced in a cell taught in U.S. Pat. Nos. 7,985,567; 8,420,833; 8,852,902; 9,109,175; 9,200,298; 9,334,514; 9,376,691; 9,382,553; 9,631,210; 9,951,345; and 10,167,488; and PCT International Patent Application Nos. PCT/US14/48293, PCT/US2018/049609, PCT/US2017/036168, PCT/US2018/029668, PCT/US2008/068833, PCT/US2008/068756, PCT/US2008/068831, PCT/US2009/042132, PCT/US2010/033299, PCT/US2011/053787, PCT/US2011/058660, PCT/US2011/059784, PCT/US2011/061900, PCT/US2012/031025, and PCT/US2013/074214 (all of which are incorporated in their entireties by reference).

### Biomass

The biomass can be obtained from one or more feedstock, such as softwood feedstock, hardwood feedstock, grass feedstock, and/or agricultural feedstock, or a mixture thereof.

Softwood feedstocks include, but are not limited to, Araucaria (e.g. A. cunninghamii, A. angustifolia, A. araucana); softwood Cedar (e.g. Juniperus virginiana, Thuja plicata, Thuja occidentalis, Chamaecyparis thyoides Callitropsis nootkatensis); Cypress (e.g. Chamaecyparis, Cupressus Taxodium, Cupressus arizonica, Taxodium distichum, Chamaecyparis obtusa, Chamaecyparis lawsoniana, Cupressus semperviren); Rocky Mountain Douglas fir; European Yew; Fir (e.g. Abies balsamea, Abies alba, Abies procera, Abies amabilis); Hemlock (e.g. Tsuga canadensis, Tsuga mertensiana, Tsuga heterophylla); Kauri; Kaya; Larch (e.g. Larix decidua, Larix kaempferi, Larix laricina, Larix occidentalis); Pine (e.g. Pinus nigra, Pinus banksiana, Pinus contorta, Pinus radiata, Pinus ponderosa, Pinus resinosa, Pinus sylvestris, Pinus strobus, Pinus monticola, Pinus lambertiana, Pinus taeda, Pinus palustris, Pinus rigida, Pinus echinata); Redwood; Rimu; Spruce (e.g. Picea abies, Picea mariana, Picea rubens, Picea sitchensis, Picea glauca); Sugi; and combinations/hybrids thereof.

For example, softwood feedstocks which may be used herein include cedar; fir; pine; spruce; and combinations thereof. The softwood feedstocks for the present invention may be selected from loblolly pine (Pinus taeda), radiata pine, jack pine, spruce (e.g., white, interior, black), Douglas fir, Pinus silvestris, Picea abies, and combinations/hybrids thereof. The softwood feedstocks for the present invention may be selected from pine (e.g. Pinus radiata, Pinus taeda); spruce; and combinations/hybrids thereof.

Hardwood feedstocks include, but are not limited to, Acacia; Afzelia; Synsepalum duloificum; Albizia; Alder (e.g. Alnus glutinosa, Alnus rubra); Applewood; Arbutus; Ash (e.g. F. nigra, F. quadrangulata, F. excelsior, F. pennsylvanica lanceolata, F. latifolia, F. profunda, F. americana); Aspen (e.g. P. grandidentata, P. tremula, P. tremuloides); Australian Red Cedar (Toona ciliata); Ayna (Distemonanthus benthamianus); Balsa (Ochroma pyramidale); Basswood (e.g. T. americana, T. heterophylla); Beech (e.g. F. sylvatica, F. grandifolia); Birch; (e.g. Betula populifolia, B. nigra, B. papyrifera, B. lenta, B. alleghaniensis/B. lutea, B. pendula, B. pubescens); Blackbean; Blackwood; Bocote; Boxelder; Boxwood; Brazilwood; Bubing a; Buckeye (e.g. Aesculus hippocastanum, Aesculus glabra, Aesculus flava/Aesculus octandra); Butternut; Catalpa; Chemy (e.g. Prunus serotina, Prunus pennsylvanica, Prunus avium); Crabwood; Chestnut; Coachwood; Cocobolo; Corkwood; Cottonwood (e.g. Populus balsamifera, Populus deltoides, Populus sargentii, Populus heterophylla); Cucumbertree; Dogwood (e.g. Cornus florida, Cornus nuttallii); Ebony (e.g. Diospyros kurzii, Diospyros melanida, Diospyros crassiflora); Elm (e.g. Ulmus americana, Ulmus procera, Ulmus thomasii, Ulmus rubra, Ulmus glabra); Eucalyptus; Greenheart; Grenadilla; Gum (e.g. Nyssa sylvatica, Eucalyptus globulus, Liquidambar styraciflua, Nyssa aquatica); Hickory (e.g. Carya alba, Carya glabra, Carya ovata, Carya laciniosa); Hornbeam; Hophornbeam; Ipe; Iroko; Ironwood (e.g. Bangkirai, Carpinus caroliniana, Casuarina equisetifolia, Choricbangarpia subargentea, Copaifera spp., Eusideroxylon zwageri, Guajacum officinale, Guajacum sanctum, Hopea odorata, Ipe, Krugiodendronferreum, Lyonothamnus lyonii (L. floribundus), Mesua ferrea, Olea spp., Olneya tesota, Ostrya virginiana, Parrotia persica, Tabebuia serratifolia); Jacarandi; Jotoba; Lacewood; Laurel; Limba; Lignum vitae; Locust (e.g. Robinia pseudacacia, Gleditsia triacanthos); Mahogany; Maple (e.g. Acer saccharum, Acer nigrum, Acer negundo, Acer rubrum, Acer saccharinum, Acer pseudoplatanus); Meranti; Mpingo; Oak (e.g. Quercus macrocarpa, Quercus alba, Quercus stellata, Quercus bicolor, Quercus virginiana, Quercus michauxii, Quercus prinus, Quercus muhlenbergii, Quercus chrysolepis, Quercus lyrata, Quercus robur, Quercus petraea, Quercus rubra, Quercus velutina, Quercus laurifolia, Quercus falcata, Quercus nigra, Quercus phellos, Quercus texana); Obeche; Okoumé; Oregon Myrtle; California Bay Laurel; Pear; Poplar (e.g. P. balsamifera, P. nigra, Hybrid Poplar (Populus×canadensis)); Ramin; Red cedar; Rosewood; Sal; Sandalwood; Sassafras; Satinwood; Silky Oak; Silver Wattle; Snakewood; Sourwood; Spanish cedar; American sycamore; Teak; Walnut (e.g. Juglans nigra, Juglans regia); Willow (e.g. Salix nigra, Salix alba); Yellow poplar (Liriodendron tulipifera); Bamboo; Palmwood; and combinations/hybrids thereof.

For example, hardwood feedstocks for the present invention may be selected from Acacia, Aspen, Beech, Eucalyptus, Maple, Birch, Gum, Oak, Poplar, and combinations/hybrids thereof. The hardwood feedstocks for the present invention may be selected from Populus spp. (e.g. Populus tremuloides), Eucalyptus spp. (e.g. Eucalyptus globulus), Acacia spp. (e.g. Acacia dealbata), and combinations thereof.

Grass feedstocks include, but are not limited to, C₄ or C₃ grasses, e.g. Switchgrass, Indiangrass, Big Bluestem, Little Bluestem, Canada Wildrye, Virginia Wildrye, and Goldenrod wildflowers, etc, amongst other species known in the art.

Agricultural feedstocks include, but are not limited to, agricultural byproducts such as husks, stovers, foliage, and the like. Such agricultural byproducts can be derived from crops for human consumption, animal consumption, or other non-consumption purposes. Such crops can be corps such as corn, wheat, sorghum, rice, soybeans, hay, potatoes, cotton, or sugarcane. The feedstock can arise from the harvesting of crops from the following practices: intercropping, mixed intercropping, row cropping, relay cropping, and the like.

In some embodiments, the biomass is an ensiled biomass. In some embodiment, the biomass is ensiled by placing the biomass in an enclosed container or room, such as a silo, or by piling it in a heap covered by an airproof layer, such as a plastic film. The biomass undergoing the ensiling, known as the silage, goes through a bacterial fermentation process resulting in production of volatile fatty acids. In some embodiment, the ensiling comprises adding ensiling agents such as sugars, lactic acid or inculants. In some embodiments, the ensiled biomass comprises one or more toxic compounds. In some embodiments, when ensiled biomass comprises one or more toxic compounds, the microbe is resistant to the one or more toxic compounds.

References cited herein include:
¹ Alder, C. M.; Hayler, J D; Henderson. R. K; Redman. A. M.; Shukla. L.; Shuster, L. E.; Sneddon, H. F. Updating and further expanding G5K's solvent sustainability guide. Green Chem., 2016, 18, 3879-3890
² Holbrey, J. D.; Rogers. R. D. Ionic liquids in synthesis; Wasserscheid, P.; Welton, T., Eds.; Wiley-VCH Verlag GmbH & Co. KGaA: Weinheim, 2002, pp 41-5.
³ Welton, T. (2011) Ionic liquids in green chemistry. Green Chemistry, 13(2), 225 https://doi.org/10.1039/c0gcU0047h
⁴ Shi, R., & Wang. Y. (2016, January). Dual ionic and organic nature of ionic liquids. Scientific Reports, 19(6), 19644.
⁵ Easton, M.; Choudhary, H.; Rogers, R. D. Azolate Anions in Ionic Liquids: Promising and Under-utilized Components of the Ionic Liquid Toolbox. Chemistry—A European Journal 2018, 25 (9), 2127-2140.
⁶ Plechkova, N. V. & Seddon, K. R. Applications of ionic liquids in the chemical industry. Chem Soc Rev 37, 123-130 (2008).
⁷Li, K.; Choudhary, H.; Rogers, R. D. Ionic Liquids for Sustainable Processes: Liquid Metal Catalysis. Current Opinion in Green and Sustainable Chemistry 2018, 11, 15-21
⁸ Choudhary, H., Li, K., Rogers, R. D. (2019). Active Pharmaceutical Ingredient Ionic Liquid: A New Platform for the Pharmaceutical Industry. In: Zhang, S. (eds) Encyclopedia of Ionic Liquids. Springer, Singapore. https://doi.org/10.1007/978-981-10-6739-6_3-1.
⁹ Biocatalysis in Ionic Liquids, Fred van Rantwijk and Roger A. Sheldon, Chem. Rev. 2007, 107, 6, 2757-2785.
¹⁰ Choudhary, H.; Berton, P.; Gurau, G.; Myerson, A. S.; Rogers, R. D. Ionic Liquids in Cross-Coupling Reactions:“Liquid” Solutions to a “Solid” Precipitation Problem. Chemical Communications 2018, 54, 2056-2059.
¹¹ Armand, M., Endres, F., MacFarlane, D. R., Ohno. H. & Scrosati, B. Ionic-liquid materials for the electrochemical challenges of the future. Nat Mater 8, 621-629 (2009).
¹² Smiglak, M.; Metlen, A.; Rogers, R D. The Second Evolution of Ionic Liquids. From Solvents and Separations to Advanced Materials—Energetic Examples from the Ionic Liquid Cookbook. Acc. Chem. Res. 2007, 40, 11, 1182-1192
¹³ Tanner, E. E. Ionic liquids charge ahead Nat Chem. 2022, 14, 842.
¹⁴ Somers, A. Howlett. P., MacFarlane. D. & Forsyth. M. A Review of Ionic Liquid Lubricants. Lubricants 1, 3-21 (2013).
¹⁵ Hapiot. P. & Lagrost, C. Electrochemical Reactivity in Room-Temperature Ionic Liquids. Chem Rev 108, 2233-2264 (2008).
¹⁶ Usmani, Z., Sharma, M., Gupta, P., Karpichev, Y., Gathergood, N., Bhat, R., et al. 12020). Ionic Liquid Based Pretreatment of Lignocellulosic Biomass for Enhanced Bioconversion Bioresour. Tech 304, 123003. doi:10.1016/j.biortech.2020.123003
¹⁷ Pham, L. T. M.; Choudhary, H.; Gauttam, R.; Singer, S. W.; Gladden, J. M.; Simmons, B. A.; Singh, S.; Sale, K. L. Revisiting Theoretical Tools and Approaches for the Valorization of Recalcitrant Lignocellulosic Biomass to Value-Added Chemicals. Front. Energy Res. 2022, 10, 863153.
¹⁸ Tadesse, H.; Luque, R. Advances on biomass pretreatment using ionic liquids: An overview. Energy Environ. Sci. 2011, 4, 3913-3929.
¹⁹ Halder, P., et al., Progress on the pre-treatment of lignocellulosic biomass employing ionic liquids. Ren. Sustain. Energy Rev. 2019, 103, 268-292.
²⁰ Zhang, J.; Zou, D.; Singh, S.; Cheng, G. Recent developments in ionic liquid pretreatment of lignocellulosic biomass for enhanced bioconversion. Sustain. Energy Fuels 2021, 5, 1655-1667.
²¹ Roy, S.; Chundawat, S. P. S. Ionic Liquid-Based Pretreatment of Lignocellulosic Biomass for Bioconversion: a Critical Review. BioEnerg. Res. 2022 https.//doi.org/10.1007?s12155-022-10425-1
²² MP Pandey, CS Kim. Lignin depolymerization and conversion: A review of thermochemical methods. Chem Eng Technol 34, 29-41 (2011).
²³ IA Pearl, Vanillin from lignin materials. J Am Chem Soc 64, 1429-1431 (1942)
²⁴ Sun, Z.; Fridrich, B.; De Santi, A.; Elangovan, S.; Barta, K. Bright side of lignin depolymerization: toward new platform. Chem. Rev. 2018, 118, 614-678.
²⁵ Chio, C.; Sain, M.; Qin. W. Lignin utilization—A review of lignin depolymerization from various aspects. Renewable Sustainable Energy Rev. 2019, 107, 232-249.
²⁶ Socha, A. M. et al., Efficient biomass pretreatment using ionic liquids derived from lignin and hemicellulose. PNAS, 2014, 111, E3587-E3595.
²⁷ Keim, M.; Kratzer, P.; Derksen, H.; Isakov, D.; Maas, G. Terminal Acetylenic Iminium Salts—Synthesis and Reactivity. Eur. J. Org. Chem. 2019, 2019, 826-844.
²⁸ Wilhelm. R. The role of iminium salts, imines and related compounds in chemistry. Z. Naturforsch, 2018, 73, 429.
²⁹ Shimizu, M.; Hachiva, I.; Mizota, I. Conjugated imnes and iminium salts as versatile acceptors of nucleophiles. Chem. Commun. 2009, 874-889
³⁰ Schiff. Hugo (1864). “Mittheilungen aus dem Universitäts-laboratorium in Pisa: 2. Eine neue Reihe organischer Basen” [Communications from the university laboratory in Pisa: 2. A new series of organic bases]. Annalen der Chemie und Pharmacie (in German). 131: 118-119. doi:10.1002/jlac.18641310113
³¹ Shamshina, J. L.; Wineinger. H. B.; Choudhary, H.; Vaid, T. P.; Kelley, S. P.; Rogers, R. D. Confusing Ions on Purpose: How Many Parent Acid Molecules Can Be Incorporated in a Herbicidal Ionic Liquid? ACS Sustainable Chem. Eng 2021, 9, 4, 1941-1948.
³² Choudhary, H.; Pernak, J.; Shamshina, J. L.; Niemczak, M., Giszter, R.; Chrzanowski, L.; Praczyk, T.; Marcinkowska, K.; Cojocaru, O. A.; Rogers, R. D. Two herbicides in a single compound. double salt herbicidal ionic liquids exemplified with glyphosate, dicamba, and MCPA. ACS Sustainable Chem Eng. 2017, 5, 6261-6273.
³³ Chemistry for Engineers, Ambasta, B. K., 2012 (4^{th} ed.), pp 295
³⁴ Yoo, C. G.; Pu. Y.; Ragauskas, A. J. Ionic liquids: Promising green solvents for lignocellulosic biomass utilization. Curr. Opin. Green Sustain. Chem 2017, 5,5-11. DOI: 10.1016/j.cogsc.2017.03.003
³⁵ van Osch, D. J. G. P.; Kollau. L. J. B. M.; van den Bruinhorst. A; Asikainen, S.; Rocha, M. A. A.; Kroon, M. C. Ionic liquids and deep eutectic solvents for lignocellulosic biomass fractionation. Phys. Chem. Chem Phys. 2017, 19, 2636-2665, DOI: 10.1039/C6CP07499E
³⁵ Xu, F., Sun, J., Konda, N V. S. N. M., Shi, J., Dutta, T., et. al., Transforming biomass conversion with ionic liquids: process intensification and the development of a high-gravity. one-pot process for the production of cellulosic ethanol. Energy Environ. Sc., 2016, 9, 1042-1049.
³⁶ Sun, J., Konda. N. V. S. N. M., Shi, J., Parthasarathi, R., Dutta. T., Xu, F., et al. (2016). CO₂ Enabled Process Integration for the Production of Cellulosic Ethanol Using Bionic Liquids. Energy Environ. Sci. 9, 2822-2834 doi:10.1039; C6EE00913A
³⁷ Sun, N., Parthasarathi, R., Socha, A. M., Shi. J., Zhang, S., Stavila, V., et al. (2014). Understanding Pretreatment Efficacy of Four Cholinium and Imidazolium Ionic Liquids by Chemistry and Computation. Green. Chem. 16, 2546-2557. doi:10.1039/c3gc42401d
³⁸ Yao, A., Choudhary, H., Mohan, M., Rodriguez, A., Magurudeniya, H., Pelton, J. G., et al. (2021). Can Multiple Ions in an Ionic Liquid Improve the Biomass Pretreatment Efficacy? ACS Sust. Chem. Eng 9, 4371-4376. doi:10.1021/acssuschemeng 0c09330
⁴⁰ Choudhary H., Simmons B A and Gladden J M (2022) Comparative Study on the Pretreatment of Aspen and Maple With 1-Ethyl-3-methylimidazolium Acetate and Cholinium Lysinate Front. Energy Res. 10:863181. doi: 10.3389; fenrg.2022.868181
⁴¹ Sun. J., Konda, N V. S. N. M, Parthasarathi, R., Dutta, T., Valley, M; Xu. F., et al. One-pot integrated biofuel production using low-cost biocompatible protic ionic liquids. Green Chem., 2017, 19, 3152-3163.
⁴² Liu, Y.-R.; Thomsen. K.; Nie, Y.; Zhang, S.-J.; Meyer. A. S. Predictive screening of ionic liquids for dissolving cellulose and experimental verification. Green Chem. 2016, 18, 6246-6254.
⁴³ Casas, A.; Palomar, J.; Alonso, M. V.; Oliet, M.; Omar, S.; Rodriguez, F. Comparison of lignin and cellulose solubilities in ionic liquids by COSMO-RS analysis and experimental validation. Ind. Crops Prod. 2012, 37, 155-163.
⁴⁴ Yang, Y.; Sharma-Shivappa, R.; Burns, J. C; Cheng, J. J. Dilute Acid Pretreatment of Oven-dried Switchgrass Germplasms for Bioethanol Production. Energy Fuels 2009, 23, 7, 3759-3766.
⁴⁵ Shi, J.; Ebrik, M. A.; Wyman, C. E. Sugar yields from dilute sulfuric acid and sulfur dioxide pretreatments and subsequent enzymatic hydrolysis of switchgrass. Biores. Technol. 2011, 102, 8930-8938.
⁴⁶ Li, C.; Knierim, B.; Manisseri, C.; Arora, R.; Scheller, H. V.; Auer, M.; Vogel. K. P.; Simmons. B. A.; Singh, S. Comparison of dilute acid and ionic liquid pretreatment of switchgrass: Biomass recalcitrance, delignification and enzymatic saccharification. Biores. Technol. 2010, 101, 4900-4906.
⁴⁷ Torr, K. M.; Love, K. T., Simmons, B. A.; Hill, S. A. Structural features affecting the enzymatic digestibility of pine wood pretreated with ionic liquids. Biotechnol. Bioeng. 2016, 113, 540-549.
⁴⁸ Jensen, A.; Cabera, Y.; Hsieh, C. W.; Nielsen, J.; Ralph, J.; Felby, C. 2D NMR characterization of wheat straw residual lignin after dilute acid pretreatment with different severities. Holzforschung, 2017, 71, 461-469.
⁴⁹ Vanholme, R.; Demedts, B.; Morreel. K.; Ralph, J.; Boerjan, W. Lignin biosynthesis and structure. Plant Phys. 2010, 153, 895-905.
⁵⁰ Abu-Omar, N. M. et al., Guidelines for performing lignin-first biorefining. Energy Environ. Sci., 2021, 14, 262-292.
⁵¹ Sluiter, A.; Hames, B.; Ruiz, R., Scarlata, C.; Sluiter, J.; Templeton, D.; Crocker, D. Determination of structural carbohydrates and lignin in biomass. Golden, CO. National Renewable Energy Labatory (NREL) Analytical Procedures 2011, TP-510-42618.
⁵² Park, S., Baker, J. O., Himmel, M. E. Parilla, P. A., Johnson, D. K Cellulose crystallinity index: measurement techniques and their impact on interpreting cellulase performance. Biotechnol Biofuels 2010, 3, 10. doi. 10.1186/1754-6834-3-10.
⁵³ Hanwell, M. D.; Curtis, D. E.; Lonie. D. C., Vandermeersch, T.; Zurek, E.; Hutchison, G. R. Avogadro: an advanced semantic chemical editor, visualization, and analysis platform. J. Cheminformatics 2012, 4, 17 (1-17).
⁵⁴ Gaussian 09, Revision D.01. Frisch, M. J.; Trucks. G. W.; Schlegel. H. B.; Scuseria, G. E.; Robb. M. A.; Cheeseman, J. R.; Scalmani, G.; Barone, V.; Mennucci, B.; Petersson, G. A.; Nakatsuji. H.; Caricato, M.; Li, X.; Hratchian, H. P.; Izmaylov, A. F.; Bloino. J.; Zheng, G.; Sonnenberg, J. L.; Hada. M.; Ehara. M.; Toyota, K.; Fukuda. R.; Hasegawa, J., Ishida, M.; Nakajima, T.; Honda. Y.; Kitao, O.; Nakai. H.; Vreven, T.; Montgomery. Jr., J. A.; Peralta, J. E.; Ogliaro, F.; Bearpark, M.; Heyd, J. J.; Brothers, E.; Kudin, K. N.; Staroverov, V. N.; Kobavashi, R.; Normand, J.; Raghavachari, K.; Rendell. A.; Burant. J. C.; lyengar, S. S.; Tomasi, J.; Cossi, M.; Rega, N.; Millam, J. M.; Klene. M.; Knox. J. E.; Cross, J. B; Bakken, V.; Adamo. C.; Jaramillo, J.; Gomperts, R.; Stratmann, R. E.; Yazyev, O.; Austin, A. J.; Cammi, R.; Pomelli, C.; Ochterski. J. W.; Martin, R. L.; Morokuma. K.; Zakrzewski. V. G.; Voth, G. A.; Salvador, P.; Dannenberg. J. J.; Dapprich, S.; Daniels, A. D.; Farkas, Ö.; Foresman. J. B.; Ortiz, J. V.; Cioslowski, J.; Fox, D. J. Gaussian, Inc., Wallingford C T, 2009.
⁵⁵ Zhang, Y.; He, H.; Dong, K., Fan. M.; Zhang, S. A DFT study on lignin dissolution in imidazolium-based ionic liquids. RSC Adv. 2017, 7, 12670-12681.
⁵⁶ Gonzalez-Miquel, M.; Massel, M.; DeSilva, A.; Palomar. J.; Rodriguez, F.; Brennecke, J. F. Excess enthalpy of monoethanolamine+ionic liquid mixtures. how good are COSMO-RS predictions? J. Phys. Chem. B 2014, 118, 11512-11522.
⁵⁷ Mohan, M.; Goud, V. V.; Banerjee, T. Solubility of glucose, xylose, fructose and galactose in ionic liquids: Experimental and theoretical studies using a continuum solvation model Fluid Phase Equilibr. 2015, 395, 33-43.
⁵⁸ Eckert, F.; Klamt, A. Fast solvent screening via quantum chemistry: COSMO-RS approach. AIChE J. 2002, 48, 364-385.
⁵⁹ Kurnia, K. A.; Pinho, S. P.; Coutinho, J. A. P. Evaluation of the Conductor-like Screening Model for Real Solvents for the prediction of the water activity coefficient at infinite dilution in ionic liquids. Ind. Eng. Chem. Res. 2014, 53, 12466-12475.
⁶⁰ Eckert, F.; Klamt, A. COSMOtherm, version C3.0 release 19.0.1. COSMOlogic GmbH & Co KG. Leverkusen. Germany, 2019.

### Example 1

### Renewable Schiff Base Ionic Liquids for Lignocellulosic Biomass Pretreatment

Growing interest in sustainable source of chemicals and energy from renewable and reliable sources stimulated the design and synthesis of renewable Schiff base (iminium) ionic liquids (ILs) to replace the fossil-derived ILs. In this study, we report the synthesis of three unique iminium-acetate ILs from lignin-derived aldehyde for a sustainable “future” lignocellulosic biorefinery. The synthesized ILs contained only imines or imines along with amines in their structure, where the ILs with only imines group exhibited better pretreatment efficacy achieving >89% sugar release. Various analytical and computational tools were employed to understand the pretreatment efficacy of these ILs. This first study demonstrating the ease of synthesis of these renewable ILs, open the door for the new class of “Schiff base ILs” for further investigation that could also be designed to be task specific.

Herein, we report, to the best of our knowledge, the first effort to protonate imines formed by direct condensation of lignin-derived vanillin with an amine such as ethylene diamine (EDA). The applicability of these iminium ILs in the processing of lignocellulosic biomass was also explored. Iminium salts are special class of organic compounds that can be visualized as □-aminocarbocations with an electrophilic nature that might also form a pseudo-base in the presence of water. Such chemical functionality could assist in unfolding several unknown interactions/chemistries when lignocellulosic biomass is considered. It must be highlighted that several examples of various iminium salts exist in the literature for various applications, 27,28,29 but the application of renewable iminium ILs in biomass processing remains unexplored until now.

### Results and Discussion

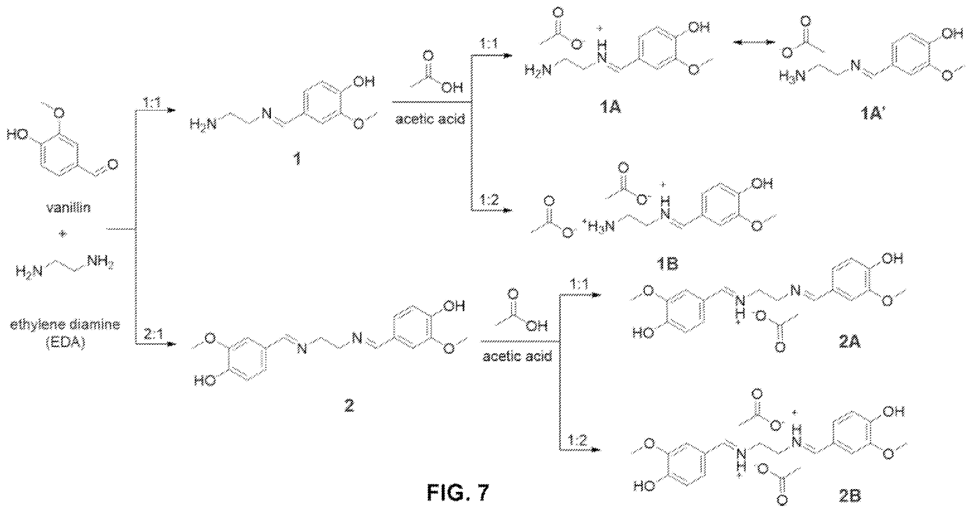

The reaction of an amine with an aldehyde (or ketone) to form an imine via carbinolamine formation were reported by Hugo Schiff in 1864 as a new series of organic bases.30 Since then these imines, also known as Schiff Bases, have been investigated in wider contexts including catalysis, and bioactive molecules. In the present study, building upon the ease of synthesis, we employed renewable lignin derived vanillin as an aldehyde precursor while ethylene diamine (EDA) as an amine source. To obtain vanillin-based Schiff bases, an aqueous solution of vanillin was slowly added to the cold aqueous solution of EDA to dissipate any immediate heat formation. The presence of two amines in EDA offers an opportunity to two unique Schiff bases 1 and 2 simply by the addition of 1 and 2 equivalents of vanillin, respectively, as indicated in FIG. 7. The products were obtained in quantitative crude yields as high melting yellow solids (melting points being >230 °C) after filtration and drying in air.

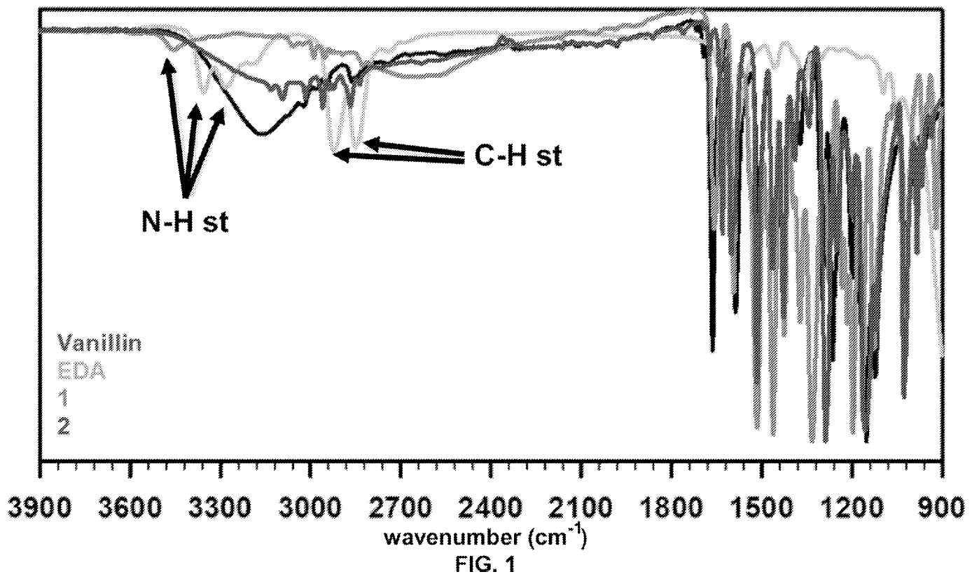

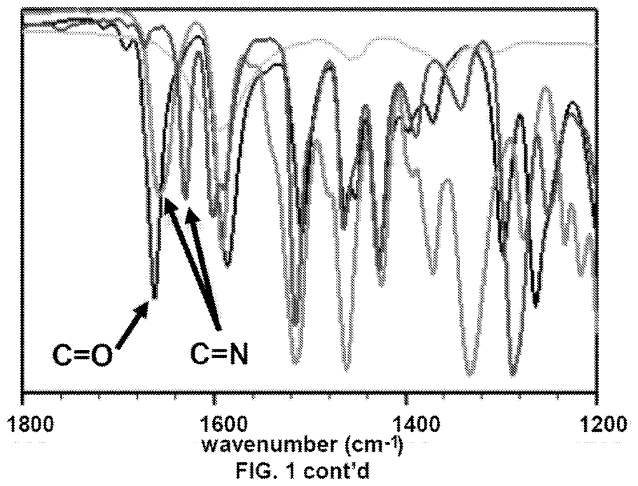

An infrared (IR) spectra of the obtained product evidenced the formation of imine bonds in 1 and 2 when compared to vanillin and EDA (FIG. 1). The N—H stretching of the primary amines weakened in 1 while completely disappearing in 2, since no primary amine is present in the molecule. Furthermore, the aromatic aldehyde (C═O) stretching @1667 cm⁻¹ in vanillin exhibits a red shift to 1641 cm⁻¹ in 1 and 1619 cm⁻¹ in 2, a signature for conjugated C═N stretching. ¹H and ¹³C NMR analysis of the synthesized Schiff bases in DMSO-d₆ suggested the formation of the desired product (see Materials and Methods).

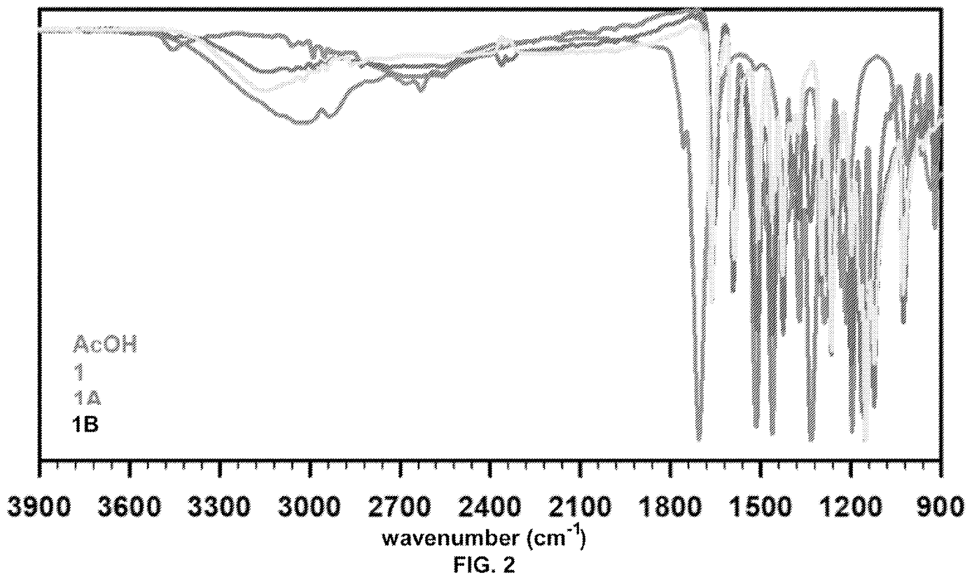

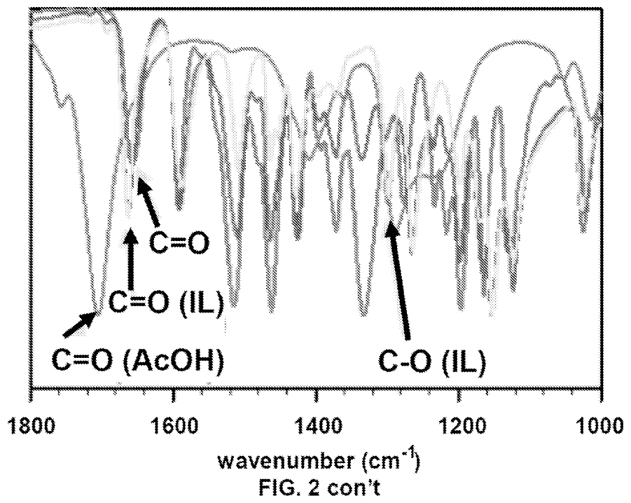

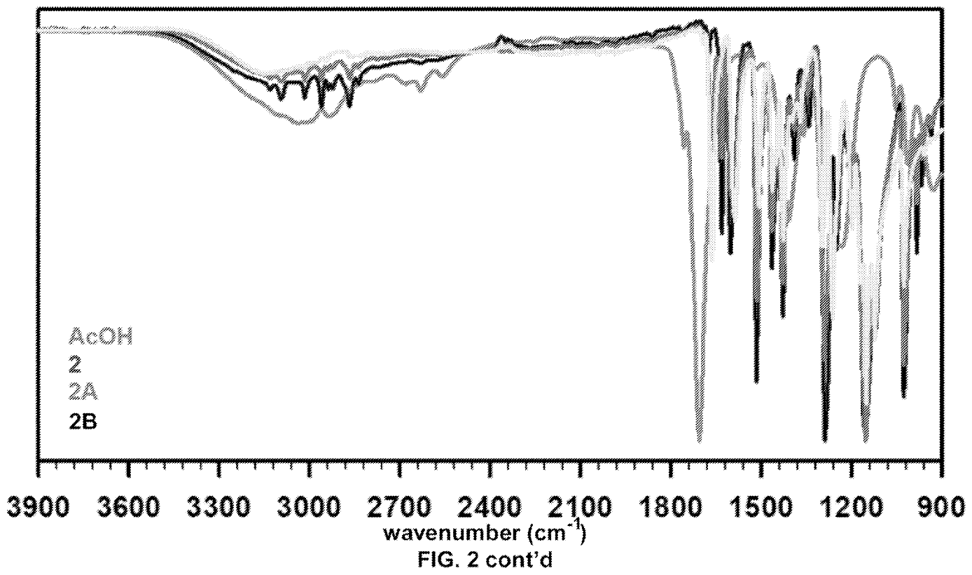

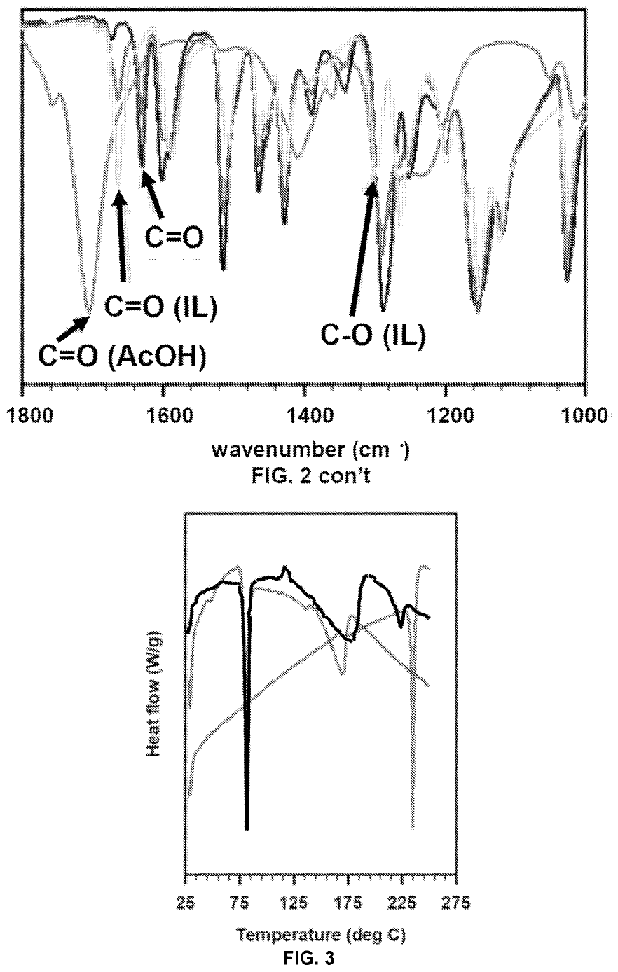

To prepare the ILs, the obtained Schiff bases 1 and 2 were then treated with acetic acid in 1:1 and 1:2 ratios to protonate the imine N-atom in these bases and yield 4 unique ILs 1A, 1B, 2A, and 2B (see FIG. 7). The synthesis protocol reflects the commonly employed acid-base reaction for the synthesis of a protic IL, where a base (imine in the present case) is mixed with an acid (acetic acid in this study) to expect proton transfer from an acid to a base (degree of proton transfer depends on the physicochemical properties of the reagents).31,32 It is important to note that acetic acid must be added slowly to cold stirred solution of Schiff bases. The rise in temperature or high concentration of acid (H+ ion) results in the hydrolysis of the C≡N bonds of Schiff bases to produce water soluble vanillin. FT-IR spectra of these ILs were recorded to understand the proton transfer (FIG. 2). The deprotonation of acid will result in a red shift of C═O stretching of carboxylic acid to the C═O stretching of carboxylate. Furthermore, the C—O stretching of carboxylate at 1296 cm⁻¹ was observed in all four IL formulations. The peaks around 2600-2700 cm⁻¹ has been assigned to strong H-bonding features in the literature.33 For instance, the intermolecular H-bonding of a concentrated acid is observed at ~2640 cm⁻¹. Such features were also observed for 1 (primary amine) but not for 2 (no primary amine). The partial protonation of amine/imine in 1 resulted in weakening of intensity in 1A while the H-bonding seems to be negligible in 1B. Similarly, the absence of free amines in 2, 2A, and 2B display no signal contributing to strong intermolecular H-bonding in these molecules. We believe due to the partial protonation of 1, a dynamic equilibrium will exist where the positive charge will fluctuate between ammonium (1A′) and iminium (1A) cations as shown in FIG. 7. The equilibrium is expected to favor protonation of amines over imines as amines are known to be more basic than imines, nevertheless we do not have any crystallographic or spectroscopic evidence at this point to support the hypothesis.

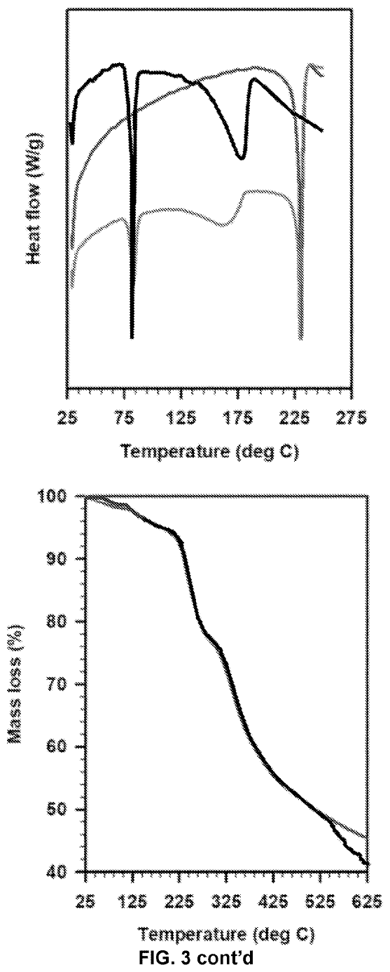

The differential scanning calorimetry (DSC) profiles of these ILs revealed the preferential proton occupancy sites in 1:1 and 1:2 acetate derivatives of 1 and 2 (FIG. 3). The high melting characteristics of imines was also observed in 1:1 acetate derivatives of these imines in the case of 2A. This indicates that 1:1 salt was not formed, instead, both nitrogen atoms in the imines seem to be fully protonated in the presence of acetic acid. When one equivalent of acetic acid was added, only 50% of the imine were fully protonated, while the rest remained neutral. This observation is consistent with the FT-IR data where the 1:1 salt displayed characteristic peaks from both imine and 1:2 acetate derivatives (see FIG. 2). We hypothesize that no- or full-protonation is not applicable for 1, since both amine (higher basicity) and imine moieties co-exist in the system. Also, thermal gravimetric analysis (TGA) curves of Schiff bases and ILs complement the DSC curves. For instance, the onset of the decomposition of the IL 2B was observed around 125 °C in both DSC and 131 TGA compared to the >200 °C for the corresponding Schiff base 2.

To explore the full chemical potential of lignocellulosic biomass, the fractionation of the strongly held constituent biopolymers (cellulose, hemicellulose, and lignin) in a complex/recalcitrant matrix thorough a pretreatment step is essential. As described earlier, due to the outstanding ability of ILs to dissolve, fractionate, and even convert biopolymers, IL-based pretreatment technologies remain attractive for a sustainable biorefinery.26,34,35,36,37 IL-based pretreatment technologies have been reported to be most effective when operated at temperatures between 90 and 160 °C for 3 h to afford high sugar yields from a given biomass.16,38,39,40,41 Next, we tested the performance of these ILs for the pre-treatment of sorghum biomass at a predetermined condition. 20 wt % of sorghum biomass was mixed with 80 wt % iminium ILs and heated at 140 °C for 3 h. It should be highlighted that all synthesized ILs were solids at room temperature, however, they are expected to melt well below the pretreatment temperature (FIG. 3). The pretreatment (PT) efficacy of these ILs are tabulated in Table 1 including solid recovery and sugar release. While considering the amount of dried biomass recovered after pretreatment with ILs followed by washing (see Materials and Methods for details), termed here as solid recovery, all ILs afforded a very high solid recovery in the range of 83-87%. Higher solid recovery at lower pretreatment temperatures are a general trend irrespective of the biomass and ILs employed for pretreatment.36,37,38,39,40,41 Snapshots of biomass mixed with ILs before and after pretreatment included in the Table 1 corroborate well with observed DSC trends, that is, IL 2A (mixture of 2 and IL 2B) did not melt completely while all others ILs melted under the pretreatment conditions. Visual observation of th results of 1A, 1B, 2A, and 2B showed that the sample is light before PT, while all of the after PT showed the samples were very dark, though 1B had the lightest color of the “after PT” samples.

TABLE 1
Pretreatment efficacy of Schiff base ILs on sorghum biomass.*

| Ionic | Solid | Sugar Release (%) | |
|---|---|---|---|
| liquid | Recovery (%) | Glucose | Xylose |
| 1A | 87.0 ± 0.96 | 62.2 ± 1.6 | 63.2 ± 1.4 |
| 2A | 83.7 ± 1.22 | 34.3 ± 0.6 | 33.4 ± 0.3 |
| 1B | 83.8 ± 2.33 | 70.0 ± 0.1 | 70.0 ± 1.9 |
| 2B | 83.4 ± 1.98 | 87.0 ± 3.6 | 76.1 ± 1.1 |

The carbohydrate (glucan and xylan) and lignin amounts of untreated and IL-pre-treated sorghum was determined to understand the impact of the pretreatment as a function of the IL. Interestingly, the carbohydrate and lignin content were found to be similar to that of the untreated biomass. For instance, the glucan, xylan, and lignin content of the IL-pretreated solids were in the range of 26.1-27.8%, 14.9-15.6%, and 19.1-21.7% respectively, compared with the 26.3% glucan, 15.1% xylan, and 19.2% lignin content in the untreated sorghum biomass. No significant loss of any biopolymer was observed after pre-treatment using these ILs even after considering the solid recovery. Typically, the actual biopolymer removal of carbohydrate and lignin component was calculated as shown below to realize a loss of <10% in all cases.

% Removal=[100−{(% solid recovery)*(composition of the pretreated biomass/composition of the untreated biomass)}]

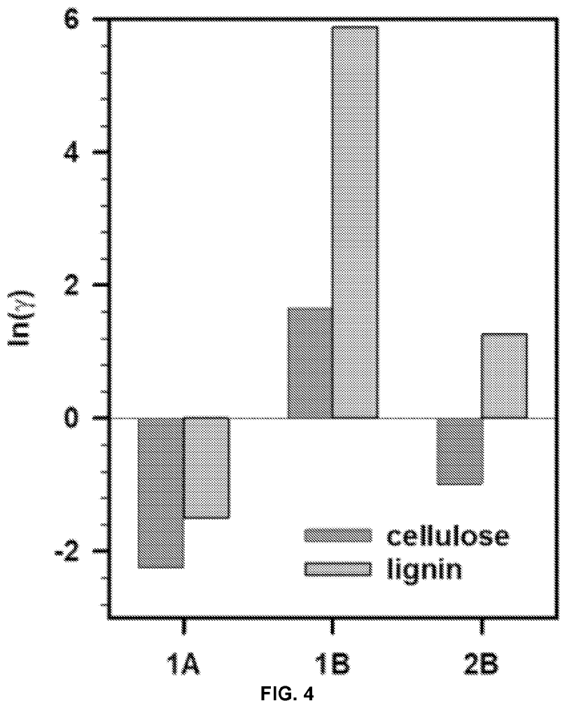

To understand these results, we performed COnductor like Screening MOdel for Real Solvent (COSMO-RS) calculations to understand the viability of IL and biopolymer interactions. In line with previous studies, we calculated the logarithmic activity coefficient (ln(7)) to predict the dissolution of biopolymers in ILs under investigation.42,43 We studied the intermolecular interactions between cellulose/lignin and ILs (FIG. 4). We did not consider IL 2A for these calculations as the composition of the synthesized IL is different and the predictions could mislead the experimental observations. In general, lower ln(7) (i.e., more negative) implies stronger interactions between the solute (cellulose or lignin) and solvent (IL). Based on these predictions, none of the ILs under investigation tend to have a significant interaction with either cellulose or lignin, especially IL 1B, which has positive ln(7) implying negligible interactions with both cellulose and lignin. In particular, all these ILs (1A and 2B) had higher affinity for cellulose over lignin. This explains the 186 negligible removal of biopolymers from the biomass after pretreatment with these ILs.

The pretreatment efficacy of these ILs was also measured in terms of carbohydrate digestibility using commercial enzymes as described in the Materials and Methods section. The enzymatic hydrolysis to release monomeric sugars from the pretreated biomass was carried out at a protein loading of 10 mg per g of biomass at 50 °C for 72 h. A high sugar release for ILs 1A, 1B, and 2B was observed affording 69-87% glucose and 63-76% xylose yields (Table 1). As expected, based on the characterization data, low sugar release was observed for IL 2A. It is worth mentioning that under similar conditions, 38% glucose and 32% xylose yields are achieved after enzymatic saccharification of pretreated solids from 20 wt % sorghum biomass and 80 wt % water mixtures at 140 °C for 3 h.

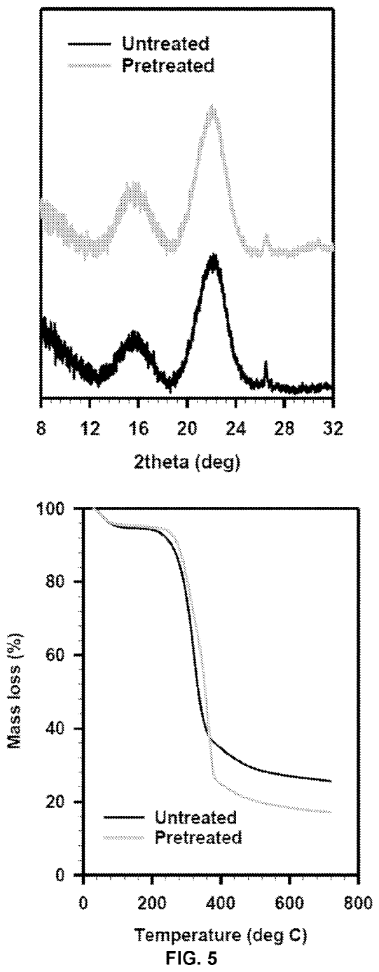

To further understand the pretreatment mechanism of the Schiff base ILs, we characterized the pretreated solids from these ILs, especially the solids after pretreatment with IL 2B as it afforded the highest sugar release (Table 1). As suggested by the COSMO-RS predictions, these ILs had higher interactions with cellulose (although negligible removal from the biomass), we recorded the powder X-ray diffraction patterns of the untreated and IL 2B pretreated sorghum to understand the interaction between cellulose and IL (FIG. 5, top right). We hypothesized that lower crystallinity of the cellulose might lead to higher enzyme accessibility, however, no such reduced crystallinity was observed for the pretreated solids. The crystallinity of the pretreated solid residue (23.9%) was similar to that of the untreated sorghum (25.5%). Additionally, the TGA of the untreated and pre-treated biomass also exhibited similar profile other than the removal of extractives including free sugars, aromatics, soluble proteins, among others (expected to be removed during washing of the pretreated biomass) (FIG. 5). Typically, most of the previous studies have either considered delignification or reduced cellulose crystallinity (cleavage of intermolecular H-bonds) to explain the pretreatment mechanism.38,39,40 While some other studies including dilute acid pretreatment reported efficient sugar release without any significant delignification or reduced crystallinity as observed in the present case.44,45,46 Also, enhanced accessible area obtained using Simons' staining and thermoporosimetry techniques were considered to explain the sugar release after pretreatment.47 Nevertheless, in the present study neither of these explains the observed pretreatment efficacy of these ILs.

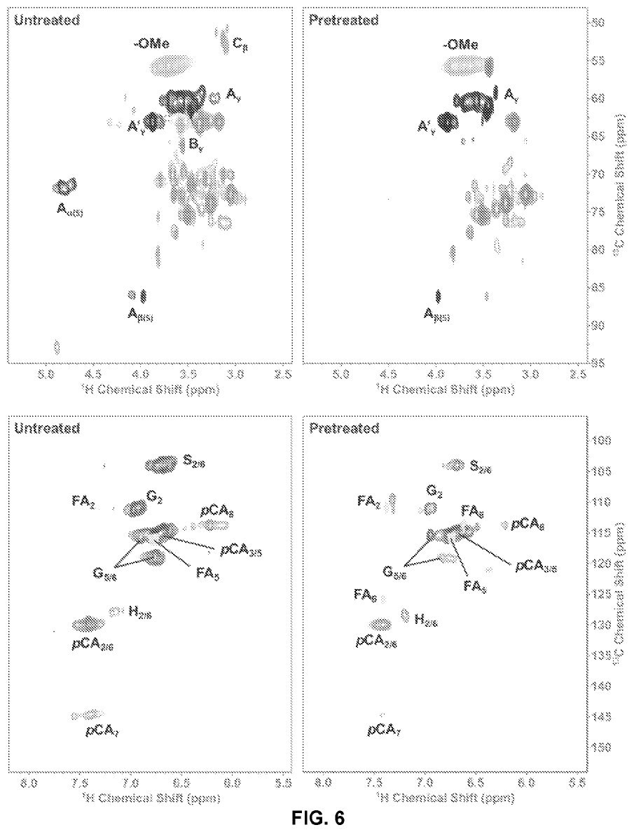

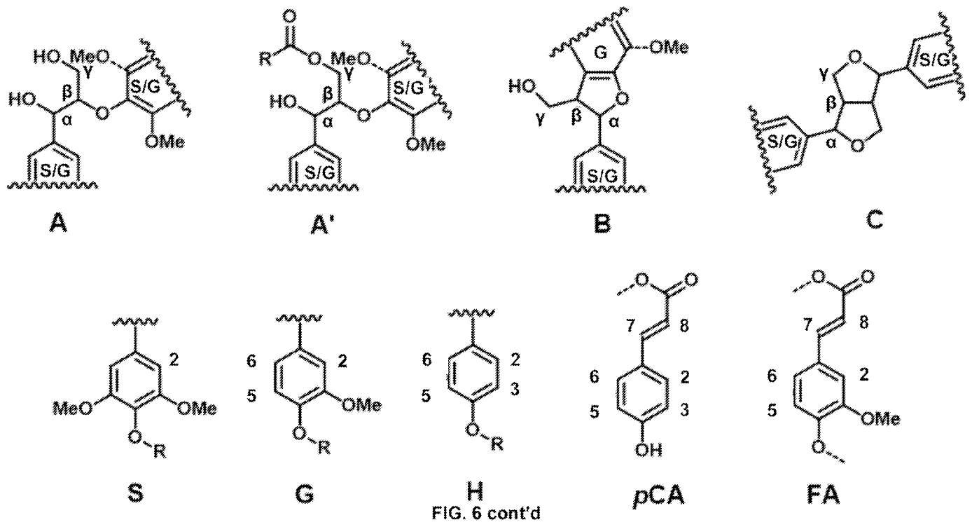

Finally, we studied the HSQC NMR of the lignin-rich residue obtained after saccharification of pretreated biomass to study the changes in structural features after pretreatment (FIG. 6). On comparison with the lignin in the untreated biomass, the observed effectiveness of the Schiff base ILs for lignocellulosic biomass pretreatment could be examined. For instance, the signals corresponding to the protons on the carbon bearing hydroxyl groups (see Aα, Aβ, Aγ, A′γ, and By structures in FIG. 6) either disappeared or weakened after pretreatment with IL 2B.48,49,50 This could be due to the chemical interaction of Schiff base IL with the lignin resulting in the abstraction of proton that leads to stripping off lignin from the recalcitrant biopolymer matrix (of cellulose, xylose, and lignin) and rendering active centers on lignin to yield condensed lignin. The aromatic region of the pretreated lignin in the HSQC NMR (FIG. 6) supported the formation of condensed lignin.48,49 These results clearly indicate the chemical interactions of Schiff Base IL with the lignocellulosic biomass resulting in high sugar yields. We propose that the Schiff base ILs investigated in this study works mainly by interacting with lignin component of the lignocellulosic biomass and not cellulose, which remains crystalline after pretreatment. However, the observation of resultant condensed lignin indicates that lignin-carbohydrate linkages in the lignocellulosic biomass was interrupted, leading to increased accessibility to the enzymes as previously reported for acid pretreatments.45 To fully exploit the specific application of the Schiff base ILs for the lignocellulosic biomass utilization for renewable fuels and products, a detailed systematic study is required to gain a better understanding of the mechanism of these ILs in lignocellulosic biomass processing.

### Materials and Methods

### Materials

All materials were used as supplied unless otherwise noted. Water was deionized, with specific resistivity of 18 M. cm at 25 °C, from Purelab Flex (ELGA, Woodridge, IL). Choline hydroxide (45% in methanol), acetic acid (>99.7%), sodium hydroxide pellets (>97%), methanol, sodium azide, and sulfuric acid (98%) were obtained from Sigma-Aldrich (St. Louis, MO). Ethanol (200 proof) was purchased from Decon Labs, Inc. (King of Prussia, PA). Sulfuric acid (72%) was procured from RICCA chemical company (Arlington, TX). J. T. Baker, Inc. (Phillipsburg, NJ) supplied hydrochloric acid and sodium citrate dihydrate, while citric acid monohydrate (>99.99%) was obtained from Merck (Kenilworth, NJ).

Analytical standard grade glucose and xylose were also obtained from Sigma-Aldrich (St. Louis, MO) and used for calibration.

Sorghum (Sorghum bicolor, donated by Idaho National Labs, Idaho Falls, USA) was dried for 24 h in a 40 °C oven. Subsequently, it was knife-milled with a 2 mm screen (Thomas-Wiley Model 4, Swedesboro, NJ). The resulting biomass was then placed in a leak-proof bag and stored in a dry cool place (4 °C room during the period of use).

Commercial cellulase (Cellic® CTec3) and hemicellulase (Cellic® HTec3) mixtures were provided by Novozymes, North America (Franklinton, NC).

### Syntheses of Schiff Base and Related Ionic Liquids

Synthesis of Schiff base. In an oven-dried round-bottomed flask (RBF) containing a Teflon-coated magnetic stirring bar, ethylene diamine was weighed and suspended in water. The flask was mounted on a cold water-bath (5 °C) and an additional funnel was attached to the RBF. An aqueous solution of vanillin was transferred to the addition funnel and added dropwise to the stirring cold aqueous solution of ethylene diamine. The mixture was then stirred for an additional 1 h. The product was obtained after filtration and drying as a yellow solid. Two different ratios of ethylene diamine to vanillin was used to get two different Schiff bases, that is 1:1 and 1:2. The purity and identity of the synthesized molecules/ILs were determined and established by NMR, IR, and thermal analysis.

Ethylene diamine—Vanillin (1:1), 1: ¹H NMR (800 MHz, DMSO-d&) S 8.56, 7.30, 7.16, 6.87, 4.01, 3.84, 2.89. ¹³C NMR (201 MHz, DMSO-d₆) δ 162.7, 151.7, 149.9, 133.4, 122.7, 118.2, 113.4, 58.1, 53.1, 40.3.

Ethylene diamine—Vanillin (1:2), 2: ¹H NMR (800 MHz, DMSO-d&) S 8.61, 7.35, 7.06, 6.84, 4.87, 3.82. ³C NMR (201 MHz, DMSO-d₆) δ 162.4, 150.9, 149.8, 132.8, 122.1, 118.7, 113.5, 60.9, 55.4.

Synthesis of ILs. In an oven-dried round-bottomed flask (RBF) containing a Teflon-coated magnetic stirring bar, known amount of Schiff bases were suspended in water. The flask was mounted on an ice-bath and an additional funnel was attached to the RBF. Acetic acid (Schiff Base to acetic acid, 1:1 and 1:2) was transferred to the addition funnel and added dropwise to the stirring cold suspension of base. The mixture was then stirred for an additional 1 h. The product was obtained after filtration and drying.

### Biomass Pretreatment

All pretreatment reactions were conducted in duplicate. 2 mm sorghum samples and IL were mixed in a 1:4 ratio (w/w) to afford a biomass loading of 20 wt % in a 15 mL capped glass pressure tube and pretreated for 3 h in an oil bath heated at 140 °C. After pretreatment, samples were removed from the oil bath and allowed to cool. 10 mL DI water-ethanol (1:1 v/v) was slowly added to the biomass-IL slurry and mixed well. The mixture was transferred to 50 mL Falcon tubes and centrifuged at high speed (4000 rpm) to separate solids and remove any residual IL. The ethanol-water washed solid was freeze-dried to obtain dried pretreated biomass for further analysis.

### Enzymatic Saccharification

All enzymatic saccharification was conducted in duplicate. Enzymatic saccharification of pretreated and untreated biomass was carried out using commercially available enzymes, Cellic® CTec3 and HTec3 (9:1 v/v) from Novozymes, at 50 °C in a rotary incubator (Enviro-Genie, Scientific Industries, Inc.). All reactions were performed at 5 wt % biomass loading in a 15 mL centrifuge tube. The pH of the mixture was adjusted to 5 with 50 mM sodium citrate buffer supplemented with 0.02% sodium azide to prevent microbial contamination. The total reaction volume included a total protein content of 10 mg per g biomass. The amount of sugars released was analyzed on an Agilent HPLC 1260 infinity system (Santa Clara, California, United States) equipped with a Bio-Rad Aminex HPX-87H column and a Refractive Index detector. An aqueous solution of sulfuric acid (4 mM) was used as the eluent (0.6 mL min-1, column temperature 60 °C).

### Compositional Analysis

All compositional analysis experiments were conducted in duplicate. Compositional analysis of biomass before and after pretreatment was performed using NREL two-step acid hydrolysis protocols (LAP) LAP-002 and LAP-005.51 Briefly, 200 mg of biomass and 2 mL of 72% sulfuric acid (H₂SO₄) were incubated at 30 °C while shaking at 200 rpm for 1 h. The solution was diluted to 4% H₂SO₄ with 56 mL of DI water and autoclaved at 121 °C for 1 h. The reaction was quenched by cooling down the flasks before removing the solids by filtration using medium-porosity filtering crucibles. The filtrates were spectrophotometrically analyzed for the acid-soluble lignin or ASL (NanoDrop 2000; Thermo Fisher Scientific, Waltham, MA) using the absorbance at 240 nm. Additionally, glucose and xylose concentrations were determined from the filtrate using HPLC (as described previously). The amount of glucan and xylan was calculated from the glucose and xylose content multiplied by the anhydro correction factors of 162/180 and 132/150, respectively. Finally, acid-insoluble lignin was quantified gravimetrically from the solid after heating overnight at 105 °C (the weight of acid-insoluble lignin and ash) and then at 575 °C for at least 6 h (the weight of ash).

### Powder X-ray Diffraction

Rigaku MiniFlex 6G 6th Generation Benchtop X-ray Diffractometer equipped with a 600W sealed source Cu tube and a HyPix-400MF Hybrid Pixel Array 0D/1D/2D detector was used for collecting powder X-ray diffraction (PXRD) data. Data collection and analysis was done with SmartLab Studio II.

The crystallinity index (CI) was determined from the crystalline and amorphous peak areas of the measured diffraction patterns using the following equation as reported previously.52

% CI=[(I₀₀₂−Iₐₘ)/I₀₀₂]*100,

where I002 is the intensity of the crystalline plane (002) and Iₐₘ is the minimum between (002) and (101) peaks and is at about 18°.

### COSMO-RS Details

Using the COSMO-RS calculations, the dissolution and/or interaction of cellulose and lignin in the Schiff base ILs was predicted. To perform these calculations, the initial structures of cellulose, lignin and ILs were drawn in the Avogadro freeware software.53 The structures of all the investigated molecules were optimized using the Gaussian09 package at B3LYP (Becke 3-parameter hybrid functional combined with the Lee-Yang-Parr correlation) theory and 6-311+G(d,p) basis set.54,55 After geometry optimization step, further, the COSMO file was generated using the BVP86/TZVP/DGA1 level of theory and basis set.56 The ideal screening charges on the molecular surface were computed using the same level of theory i.e., BVP86 through the “scrf=COSMORS” keyword.57 The generated COSMO files were then used as an input in the COSMOtherm (version 19.0.1, COSMO-logic, Leverkusen, Germany) package with BP_TZVP_19 parametrization.58 In COSMORS calculations, the molar fraction of lignin was set as 0.2, whereas the molar fraction of solvents was set to 0.8 to mimic the experimental pretreatment setup with a biomass to IL loading ratio of 1:4 (w/w). The activity coefficient of component i is associated with the chemical potential μi and expressed as,59

$$ln ( {γ}_{i} ) = ( \frac{{μ}_{i} - {μ}_{i}^{0}}{RT} )$$

where, μi⁰ is the chemical potential of the pure component i, R is the real gas constant and Tis the absolute temperature. The details of COSMO-RS calculation are provided in the COSMOtherm's user manual.60

### FT-IR Analysis

The identity of Schiff base and related ILs was established using FT-IR spectroscopy using a Bruker VERTEX 70/80 system (Billerica, MA). The data was analyzed using OPUS (version 8.2, build 8, 2, 28 (20190310) software.

### Thermal Analysis

Thermal behavior was determined using a Mettler Toledo Stare TGA/DSC1 unit (Mettler Toledo, Leicester, UK) under nitrogen (50 mL/min). Samples between 3 and 10 mg were placed in alumina crucibles (70 μL) and heated from room temperature to 800 °C at a heating rate of 10 °C/min to obtain thermal decomposition profiles. Similarly, the Schiff bases and related ILs were sealed in a Hermetic Al pan and the heated from room temperature to 250 °C at a heating rate of 10 °C/min to obtain thermal transition profiles. The data was analyzed using STARe Evaluation software.

### HSQC NMR

Untreated and pretreated biomass obtained after enzymatic saccharification were ground with a mixer mill (Qiagen MM300 Mixer, Retsch) using 2 mm diameter stainless steel balls and 30 s⁻¹ mixing frequency for 15 min. The ground material was dispersed in DMSO-d₆ and allowed to stand overnight to extract lignin. The 2D heteronuclear single quantum coherence (HSQC) spectra were collected on a Bruker Avance I 800 MHz spectrometer equipped with a TXI probe at 310K. A standard Bruker pulse sequence (hsqcetgpsisp2.2) was used with the following parameters which are typical for plant cell wall samples. HSQC spectra were collected from 11 to −1 ppm in F2 (¹H) dimension with 1024 data points for 53 ms acquisition time, and from 165 to −10 ppm in F1 (¹³C) dimension with 256 data points for 3.5 ms acquisition time. A total of 256 scans were recorded for each t1 point with a pulse delay of 1 s. The central DMSO solvent peak was used as a reference for the chemical shift calibration for all samples (δC 39.5 ppm, δH 2.5 ppm). All HSQC spectra were processed using typical 90° sine square apodization in both F2 and F1 dimensions and the contours were integrated in the MestreNOVA software (v.14). Peaks were assigned according to published data.

### CONCLUSIONS

In summary, we have developed lignin-based renewable Schiff base ILs and explored their application in the lignocellulosic biomass pretreatment. It was noted that imines preferred to be completely protonated rather than being in a dynamic equilibrium of proton hoping from one iminium center to the other. Also, the fully protonated iminium IL 2B was most effective in affording highest glucose (~87%) and xylose (~76%) release—although negligible interactions with biopolymers was realized based on experimental (no significant removal of biopolymers after water wash) and simulated data. Interestingly, HSQC NMR spectra suggested changes in the lignin structure after pretreatment with IL 2B implying interactions between IL and biopolymers. We would like to emphasize that this work demonstrates a single example of the large number of lignin-derived aldehyde and amine combinations that can be designed and applied for a range of applications including lignocellulosic biomass pretreatment, enabling an overall lower environmental and economic impact. Additionally, rigorous technoeconomic and life cycle models are essential to understand the overall impact and best-suited application of the new class of Schiff base ILs.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method to deconstruct a biomass: the method comprising: (a) introducing a solvent comprising a Schiff-base ionic liquid (SBIL) to a biomass, such that the solvent solubilizes at least a part of the biomass to form a solubilized biomass mixture.

2. The method of claim 1, further comprising: (b) separating a solubilized biomass from the SBIL; (c) optionally introducing an enzyme and/or a microbe to the solubilized biomass such that the enzyme and/or microbe produces a sugar from the solubilized biomass; and, (d) optionally separating the sugar from the solubilized biomass.

3. The method of claim 1, further comprising: forming the SBIL prior to the introducing step, and optionally forming the solvent by providing the SBIL.

4. The method of claim 1, further comprising: ensiling the biomass prior to the introducing step (a).

5. The method of claim 1, wherein the SBIL are derived or obtained from lignin.

6. A composition comprising: (a) a solvent comprising a Schiff-base ionic liquid (SBIL), and (b) a biomass.

7. The composition of claim 6, wherein the SBIL comprises a C═N moiety formed by condensation of an aldehyde or ketone and an amine.

8. The composition of claim 7, wherein the SBIL is an azomethine.

9. The composition of claim 6, wherein the solvent further comprises an ionic liquid (IL) that is not a Schiff-base, and/or a deep eutectic solvent (DES).

10. The composition of claim 6, wherein the SBIL comprise a vanillin and an ethylenediamine.

11. The composition of claim 10, wherein the vanillin and the ethylenediamine have a molar ratio of about 1:1 to about 2:1.

12. The composition of claim 6, wherein the biomass comprises a biomass from sorghum.

13. The composition of claim 6, comprising 20 wt % biomass and 80 wt % solvent.
